(19) United States
(12) Patent Application Publication
(10) Pub. No.: US 2015/0170381 A1
(43) Pub. Date: Jun. 18, 2015

(54) TOOL LOCALIZATION SYSTEM WITH IMAGE ENHANCEMENT AND METHOD OF OPERATION THEREOF

(71) Applicant: Sony Corporation, Tokyo (JP)

(72) Inventors: Ming-Chang Liu, San Jose, CA (US); Liangyin Yu, Fremont, CA (US); Seiji Kobayashi, Tokyo (JP)

(21) Appl. No.: 14/107,886

(22) Filed: Dec. 16, 2013

(51) Int. Cl. G06T 11/00; G06T 7/00; H04N 5/232

(52) U.S. Cl. CPC G06T 11/001; H04N 5/23229; G06T 7/0012; G06T 7/0046; G06T 7/0081

(57) ABSTRACT

A tool localization system and method of operation thereof including: a camera for obtaining an image frame; and a processing unit connected to the camera, the processing unit including: a classification module for detecting a surgical tool in the image frame, a motion vector module, coupled to the classification module, for modeling motion of the surgical tool based on the image frame and at least one prior image frame, a mask generation module, coupled to the motion vector module, for generating a tool mask, based on the surgical tool detected and the motion of the surgical tool, for covering the surgical tool in the image frame, and an exposure module, coupled to the mask generation module, for processing the image frame without the areas covered by the tool mask for display on a display interface.

### TECHNICAL FIELD

The present invention relates generally to a tool localization system, and more particularly to a system for enhancing an image with tools in the image.

### BACKGROUND ART

Advances in medical technology have improved recovery times and reduced complication rates. One significant advance is the increasing prevalence of laparoscopic surgery, which avoids the need for cutting large holes in a patient for surgery by using small incisions to insert tools and a camera (i.e., endoscope or laparoscope) so the surgeon can see inside the patient. The ability for a surgeon to easily see the operation space is paramount to the success of the surgery.

However, the images from an endoscopic or laparoscopic camera can be of low quality due to a number of issues such as over- or under-exposure, insufficient light, condensation, bodily fluids obscuring the lens, or other problems.

Thus, a need still remains for obtaining a better image from inside a patient. In view of the ever-growing importance of healthcare, it is increasingly critical that answers be found to these problems. In view of the ever-increasing commercial competitive pressures, along with growing consumer expectations and the diminishing opportunities for meaningful product differentiation in the marketplace, it is critical that answers be found for these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

The present invention provides a method of operation of a tool localization system including: obtaining an image frame with a camera; detecting a surgical tool in the image frame; modeling motion of the surgical tool based on the image frame and at least one prior image frame; generating a tool mask, based on the surgical tool detected and the motion of the surgical tool, for covering the surgical tool in the image frame; and processing the image frame without the areas covered by the tool mask for display on a display interface.

The present invention provides a tool localization system, including: a camera for obtaining an image frame; and a processing unit connected to the camera, the processing unit including: a classification module for detecting a surgical tool in the image frame, a motion vector module, coupled to the classification module, for modeling motion of the surgical tool based on the image frame and at least one prior image frame, a mask generation module, coupled to the motion vector module, for generating a tool mask, based on the surgical tool detected and the motion of the surgical tool, for covering the surgical tool in the image frame, and an exposure module, coupled to the mask generation module, for processing the image frame without the areas covered by the tool mask for display on a display interface.

Certain embodiments of the invention have other steps or elements in addition to or in place of those mentioned above. The steps or element will become apparent to those skilled in the art from a reading of the following detailed description when taken with reference to the accompanying drawings.

### BRIEF DESCRIPTION OF THE DRAWINGS

FIG. 1 is a schematic of a tool localization system in an embodiment of the present invention.

FIG. 2 is an exemplary image frame displayed on the display interface of FIG. 1.

FIG. 3 is the exemplary image frame of FIG. 1 in an image processing phase of operation.

FIG. 4 is another exemplary image frame in a classification phase of operation.

FIG. 5 is the another exemplary image frame of FIG. 4 in a tool isolation phase of operation.

FIG. 6 is a few examples of tool shape templates for use in a tool shape modeling phase of operation.

FIG. 7 is yet another exemplary image frame in a motion modeling phase of operation.

FIG. 8 is the yet another exemplary image frame of FIG. 7 in a motion tracking phase of operation.

FIG. 9 is an image processing flow chart detailing the tool isolation and tool shape modeling phases of operation.

FIG. 10 is a flow chart of a method of operation of the tool localization system in a further embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known circuits, system configurations, and process steps are not disclosed in detail.

The drawings showing embodiments of the system are semi-diagrammatic and not to scale and, particularly, some of the dimensions are for the clarity of presentation and are shown exaggerated in the drawing FIGS. Similarly, although the views in the drawings for ease of description generally show similar orientations, this depiction in the FIGS. is arbitrary for the most part. Generally, the invention can be operated in any orientation.

Where multiple embodiments are disclosed and described having some features in common, for clarity and ease of illustration, description, and comprehension thereof, similar and like features one to another will ordinarily be described with similar reference numerals. The embodiments have been numbered first embodiment, second embodiment, etc. as a matter of descriptive convenience and are not intended to have any other significance or provide limitations for the present invention.

For expository purposes, the term “horizontal” as used herein is defined as a plane parallel to the plane or surface of the bottom of an image frame. The term “vertical” refers to a direction perpendicular to the horizontal as just defined. Terms, such as “above”, “below”, “bottom”, “top”, “side” (as in “sidewall”), “higher”, “lower”, “upper”, “over”, and “under”, are defined with respect to the horizontal plane, as shown in the figures. The term “on” means that there is direct contact between elements. The term “directly on” means that there is direct contact between one element and another element without an intervening element.

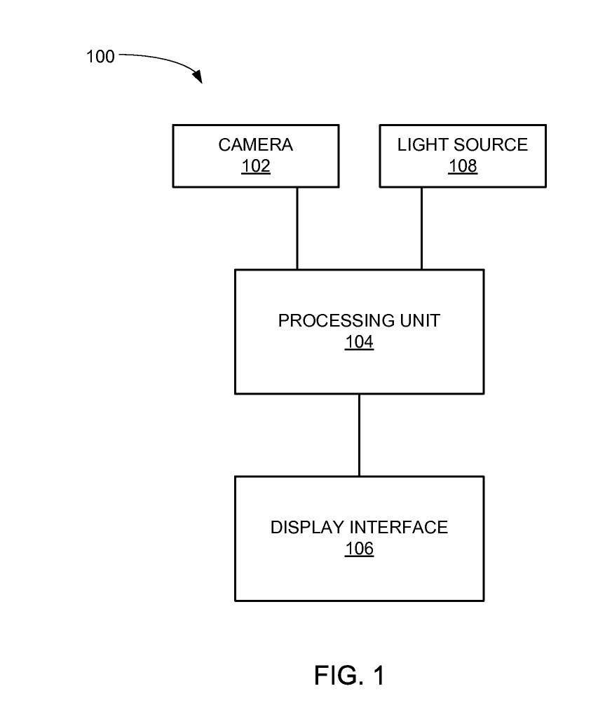

Referring now to FIG. 1, therein is shown a schematic of a tool localization system 100 in an embodiment of the present invention. Shown are a camera 102, a processing unit 104, and a display interface 106.

The camera 102 can be a camera capable of capturing video. The camera 102 is connected to the processing unit 104, which is connected to the display interface 106. The display interface 106 displays the view of the camera 102. Also connected to the processing unit 104 is a light source 108 for illuminating objects in view of the camera 102. The processing unit 104 is shown as connected to the light source 108 for illustrative purposes, and it is understood that the light source 108 can also be separate from the processing unit 104.

The processing unit 104 can be any of a variety of semiconductor devices such as a general purpose computer, a specialized device, embedded system, or simply a computer chip integrated with the camera and/or the display interface 106. The display interface 106 can utilize a variety of display technologies such as LCD, LED-LCD, plasma, holographic, OLED, front and rear projection, CRT, or other display technologies.

The processing unit 104 can contain many modules capable of performing various functions. For example, the processing unit 104 can have classification module containing a tissue modeling module coupled to a boundary detection module, a template comparison module coupled to the classification module, a motion vector module coupled to a motion tracking module, with both coupled to the template comparison module and the mask generation module. The processing unit can run some or all of the modules simultaneously.

For example, the camera 102 can be used in conjunction with the light source 108 and surgical tools in order to perform laparoscopic surgery which is also known as minimally invasive surgery. The camera 102, the light source 108, and the surgical tools can be inserted into a patient, with the display interface 106 showing a view from the camera 102 illuminated by the light source 108 of the area to be manipulated with the surgical tools. Laparoscopic surgery is given as an example of how the tool localization system 100 can be used, but it is understood that the tool localization system 100 can be used in different contexts. For example, the tool localization system 100 can be integrated into a handheld camera, phone, or tablet, or operated as a camera attached to a personal computer or laptop.

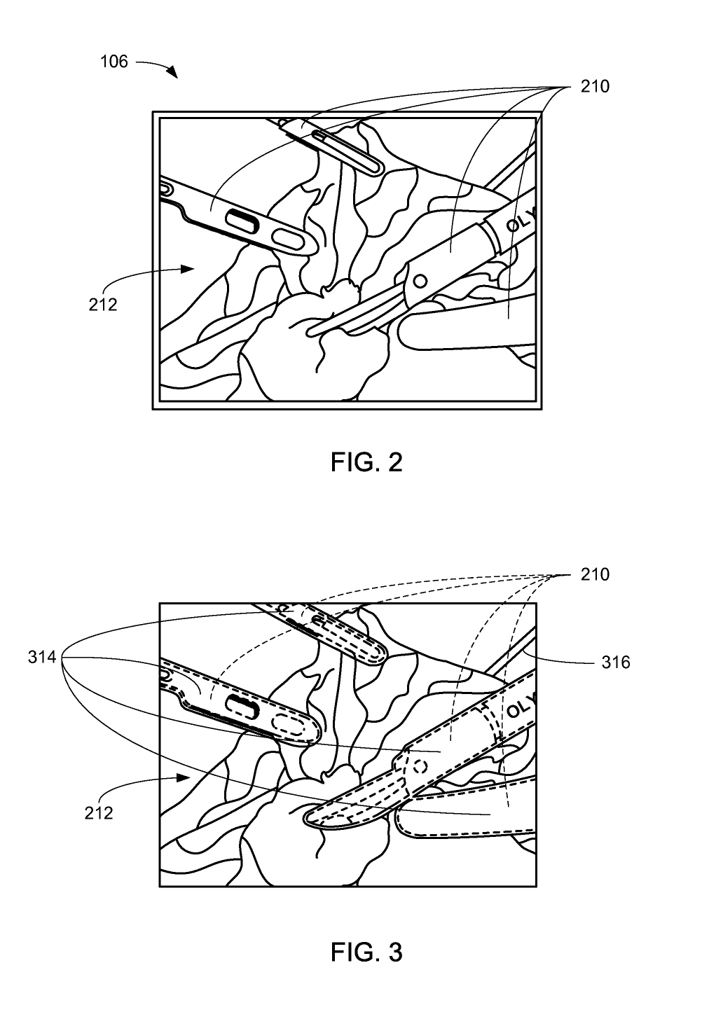

Referring now to FIG. 2, therein is shown an exemplary image frame displayed on the display interface 106. Seen here are surgical tools 210 and a background of interest 212, such as human tissue. This figure is an exemplary view of what is seen during laparoscopic surgery inside a patient. In this exemplary view can be seen blood vessels and other features of interest (as squiggly and wavy lines) of the background of interest 212 for manipulation by the surgical tools 210.

The view shown represents a properly exposed image frame wherein features of the background of interest 212 along with the surgical tools 210 are easily seen. “Exposed” or “exposure” as used herein is defined as relating to the photographic term “exposure” which generally references the amount of light the camera captures. For example, “underexposed” refers to an image where there is loss of detail in dark areas, and “overexposed” refers to an image where there is loss of detail in bright areas.

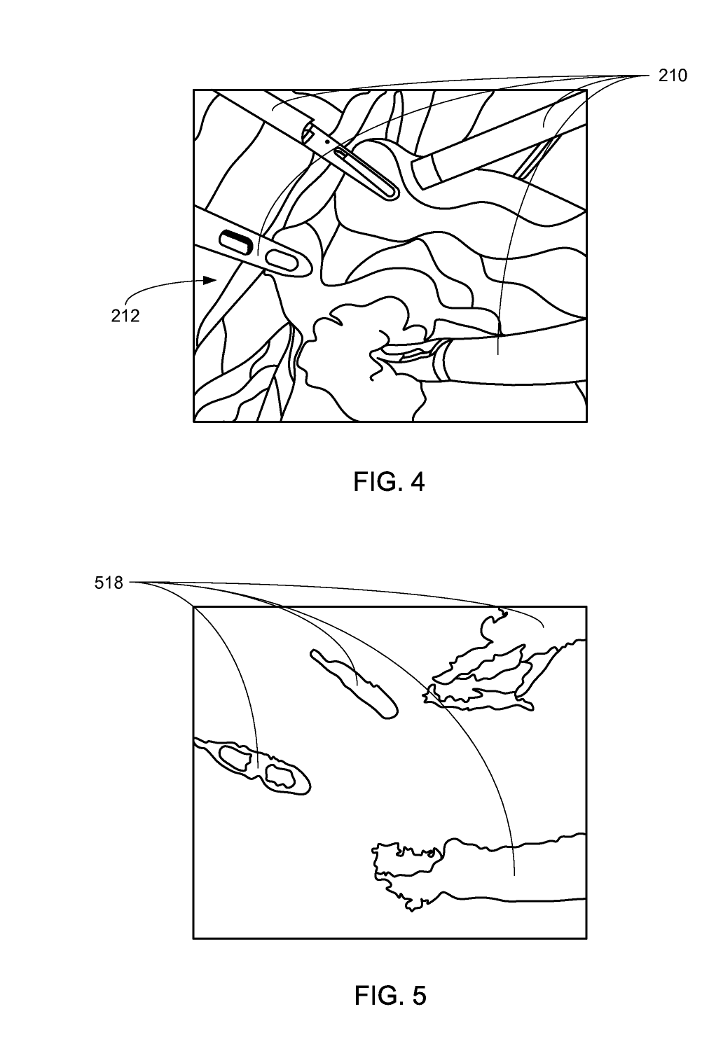

Referring now to FIG. 3, therein is shown the exemplary image frame of FIG. 1 in an image processing phase of operation. In order to obtain a properly exposed image frame wherein features of the background of interest 212 are easily seen, the image frame is processed for exposure measure (checking for average light level to properly set exposure) with the surgical tools 210 represented, replaced, or covered with a tool mask 314 which is excluded from the image frame during measurements to calculate proper exposure settings.

The surgical tools 210 in this view are shown with dotted lines for illustrative purposes only because the surgical tools 210 are masked or covered by the tool mask 314, which follows the contours of the shape of the surgical tools 210. This effectively removes the surgical tools 210 from the image frame during exposure calculations. Because most kinds of the surgical tools 210 are metallic and highly reflective as compared to the background of interest 212 (the tissue being operated on), and exposure setting is generally done on the entire image frame, bright spots (reflections off of the surgical tools 210 from the light source 108, for example) can throw off the exposure calculation. Thus, the bright spots from the surgical tools 210 can lead to underexposing the image frame, which can cause darker areas of the background of interest 212 to lose detail, and lead to sub-optimal image quality.

It has been discovered that using the tool mask 314 to remove the surgical tools 210 from the image frame for purposes of exposure measure produces better image quality. For example, because good image quality for the background of interest 212 is paramount for ease of surgery, the tool mask 314 removing the brightness of the surgical tools 210 from the exposure measurements can lead to better exposure (more accurate picture, more detail) of the background of interest 212.

It has also been discovered that the tool mask 314 covering most, but not all, of the surgical tools 210 still produces good image quality. For example, because exposure is generally taken from the average brightness of a given image, a few unmasked portions of the surgical tools 210 should not significantly affect image quality. Visible in this example is an example of a portion of an unmasked surgical tool 316 (seen at the top right of the image frame) which is a small percentage of the frame, and for the purposes of this example, is also largely in shadow; this should generate fewer exposure-skewing reflections.

It has also been found that the tool mask 314 can be used to improve other types of image processing aside from exposure measure. For example, the tool mask 314 removing the surgical tools 210 from the image frame when processing the image can improve resolution and picture quality when using other types of electromagnetic radiation other than visible light. Also for example, the tool mask 314 can replace the surgical tools 210 and be shown on the display interface 106 of FIG. 1 as translucent or largely transparent “ghost” outlines over the background of interest 212, which can allow full view of the background of interest 212 unobstructed by the surgical tools 210 while allowing a viewer to continue to operate the surgical tools 210 guided by the translucent tool outlines.

Referring now to FIG. 4, therein is shown another exemplary image frame in a classification phase of operation. In this figure, and other following figures, the position and sometimes shape of the surgical tools 210 and the content of the background of interest 212 are different from the exemplary image frame of FIG. 2, but it is understood that this is for illustrative purposes only. It is understood that the same image frame can go through every step of operation of the tool localization system 100. It is also understood that the classification and motion tracking of the surgical tools 210 can be done on any variety of shapes and types of the surgical tools 210 without limitation to the types or shapes shown in the figures.

In the another exemplary image frame can be seen the background of interest 212 and the surgical tools 210. This figure shows an example of a base or raw image frame for later processing. Also seen in this image frame are the same squiggly and wavy lines representing blood vessels and tissue boundaries of the human tissue of the background of interest 212.

Referring now to FIG. 5, therein is shown the another exemplary image frame of FIG. 4 in a tool isolation phase of operation. Shown are potential tool outlines 518 isolated from the background of interest 212 of FIG. 4. The another exemplary image frame can first be processed through segmentation, edge detection, boundary detection, and/or line detection steps to separate and group pixels of the image frame, for example. Lines detected in the image frame can be considered to be boundaries, and the areas defined by the boundaries can be compared against known patterns. The potential tool outlines 518 shown are for example only, and illustrate the difficulty of detecting even straight lines against the noisy background of human tissue.

For example, human tissue models (known appearance of particular types of tissue, for example) can be used to identify the background of interest 212, which can then excluded from the search for the potential tool outlines 518. Remaining areas within detected boundaries can be processed by utilizing known tool templates compared against outlined areas of the segmented image frame.

Because the surgical tools 210 of FIG. 4 (and surgical tools in general) share some general characteristics such as consistent color (metallic), a generally elongated shape, and a rigid body, a preliminary tool isolation process can outline all of the potential surgical tools in the image frame. The potential tool outlines 518 mark groups of pixels of interest for later motion modeling to determine which of the potential tool outlines 518 truly correspond to the locations of the surgical tools 210. The entire set of pixels or a portion of the pixels in the potential tool outlines 518 may be found to be the surgical tools 210. For example, because the surgical tools 210 each have a rigid body, that means that if the pixels or a portion of the pixels of one of the potential tool outlines 518 moves as a unit, there is a high chance one of the surgical tools 210 has been isolated.

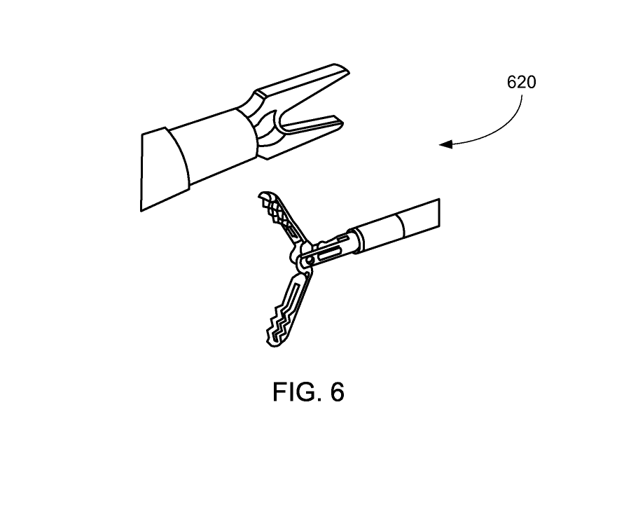

Referring now to FIG. 6, therein is shown a few examples of tool shape templates 620 for use in a tool shape modeling phase of operation. Before checking to see if the potential tool outlines 518 of FIG. 5 move as a unit, the shapes of the potential tool outlines 518 from the correct angle can be compared against the tool shape templates 620 to look for a strong match. Such a match will strongly indicate that the particular one of the potential tool outlines 518 that matches with a particular one of the tool shape templates 620 should be investigated for motion modeling. Additionally, the tool shape templates 620 can be used to help generate the potential tool outlines 518, with a cross-check against movement consistency (for example, movement as a unitary body) to ensure accurate generation of the potential tool outlines 518.

The tool shape templates 620 shown are for example only, and it is understood that as many of the tool shape templates 620 as are necessary can be stored. The tool shape templates 620 also can contain enough information to take into account the three-dimensional shape of the surgical tools 210 of FIG. 4.

It has been discovered that having three-dimensional information about the surgical tools 210 allows for more effective tool detection and isolation. For example, this three-dimensional information should allow the tool localization system 100 of FIG. 1 to detect and isolate the surgical tools 210 from the background of interest 212 of FIG. 4 no matter the orientation or angle of the surgical tools 210 relative to the camera 102 of FIG. 1 and the light source 108 of FIG. 1.

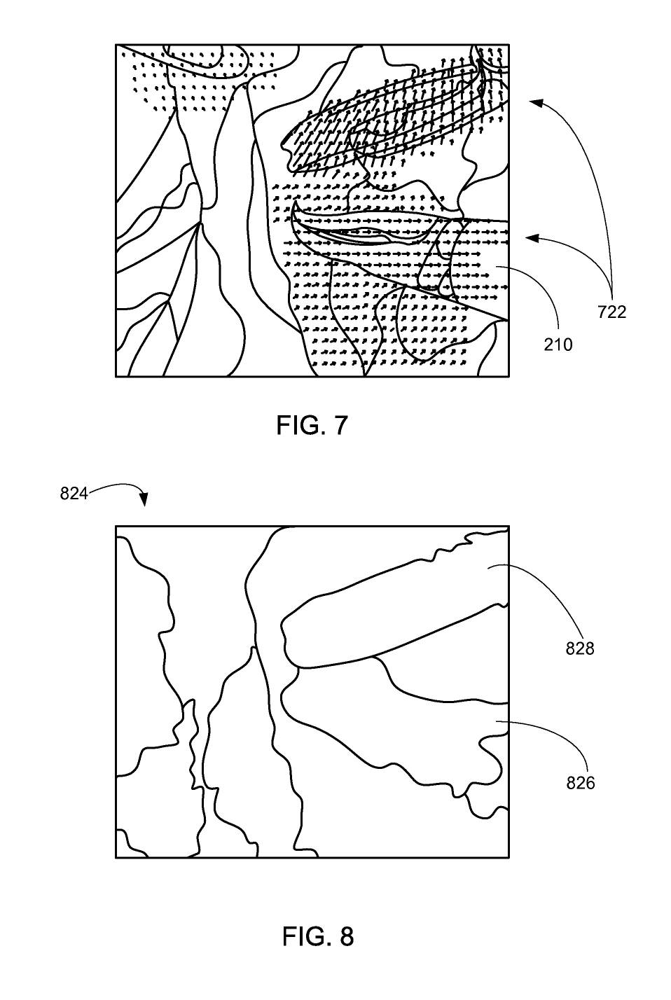

Referring now to FIG. 7, therein is shown yet another exemplary image frame in a motion modeling phase of operation. Shown are other examples of the surgical tools 210 and the background of interest 212, along with a motion vector overlay 722. Only one of the surgical tools 210 is labeled for clarity. The motion vector overlay 722 is shown as arrows in a grid overlaying the surgical tools 210, and can represent the movement of pixels or groups of pixels in the image frame. The arrows are shown as overlaying the surgical tools 210 because in this example the largest amount of movement will be of the surgical tools 210, but it is understood that the motion vector overlay 722 can be over any part of the image frame.

The motion vector overlay 722 can be calculated by comparing a number of previous or prior captured image frames to a current image frame, for example. At least one prior image frame and the current image frame can be used to calculate or generate the motion vector overlay 722. The arrows of the motion vector overlay 722 are shown spaced such that the arrows are clearly visible, but it is understood that the motion vector overlay 722 can be generated at higher or lower resolutions as necessary. For example, the motion vector overlay 722 can be generated on a per pixel basis if such level of resolution is necessary.

The motion vector overlay 722 can be combined with the potential tool outlines 518 of FIG. 5 and the tool shape templates 620 of FIG. 6 to determine what portions of the image frame are the surgical tools 210. This process can be performed in various ways. For example, as described earlier, the tool shape templates 620 can be compared to the potential tool outlines 518 to make a preliminary determination as to the locations of the surgical tools 210, but accuracy can be increased by using the motion vector overlay 722.

Continuing the example, the surgical tools 210 can be isolated if two conditions are met, for example. First, the motion vector overlay 722 shows that one of the potential tool outlines 518 or a portion of one of the potential tool outlines 518 matches up with one of the tool shape templates 620; the number of matching pixels exceeding a threshold pixel percentage match, for example. Second, the potential match can be compared to the motion vector overlay 722 to see whether the potential tool outlines 518 matched with the tool shape templates 620 are moving as a rigid body (moving as a single unit in translation and rotation); that is, the pixels within the potential tool outlines 518 are associated with vectors in the motion vector overlay 722 that are all pointing in the same direction and consistent with a unitary object, for example.

Referring now to FIG. 8, therein is shown the yet another exemplary image frame of FIG. 7 in a motion tracking phase of operation. Once the surgical tools 210 of FIG. 7 can be isolated from the rest of the image frame, a motion tracking layer 824 having prioritized tracking sectors 826 can be generated to speed up processing time and improve tracking of the surgical tools 210.

The motion tracking layer 824 can be generated in a number of ways. For example, the various vectors of the motion vector overlay 722 of FIG. 7 can be grouped based on correlation with the potential tool outlines 518 of FIG. 5. This can be followed by the areas of the image frame being assigned priority values based on the strength of correlation. For example, when there is a high correlation between the grouped vectors of the motion vector overlay 722, the area covered by said grouped vectors can be designated as one of the prioritized tracking sectors 826. The prioritized tracking sectors 826 can be given different levels of tracking priority based on the strength of correlation, for example. As a further example, the prioritized tracking sectors 826 can be color-coded to correspond to tracking priority. In this example, high priority tracking sectors 828 are designated at areas that correspond to some of the surgical tools 210 of FIG. 7.

The prioritized tracking sectors 826, the potential tool outlines 518, and the motion vector overlay 722 (for motion prediction, for example) can be combined to generate the tool mask 314 of FIG. 3, which can be used in the manner previously described to mask out the surgical tools 210 in order to properly set exposure levels to obtain the greatest level of detail when looking at the background of interest 212 of FIG. 2, for example. Through use of the motion vector overlay 722, the potential tool outlines 518, and the prioritized tracking sectors 826, the tool mask 314 can track the movement of the surgical tools 210 as the surgical tools 210 move around within the field of view of the camera 102 of FIG. 1. For example, the prioritized tracking sectors 826 can be used to modify processing of a subsequent image frame and improve processing speed by weighting certain boundaries more if they fall within the prioritized tracking sectors 826.

It has been discovered that the use of the prioritized tracking sectors 826 in conjunction with the potential tool outlines 518 can improve usability of the tool localization system 100. For example, the prioritized tracking sectors 826 can allow prioritized processing of the image frame for certain sectors rather than the entire image, which can speed processing of the image frame that is eventually shown on the display interface 106 of FIG. 1. Processing the entire image frame every time could lead to delay or lag between what the camera sees and what is shown on the display interface 106. Reducing this lag by reducing the latency or processing time between when the frame is first captured and finally displayed, a surgeon or user will find the tool localization system 100 easier and more intuitive to use.

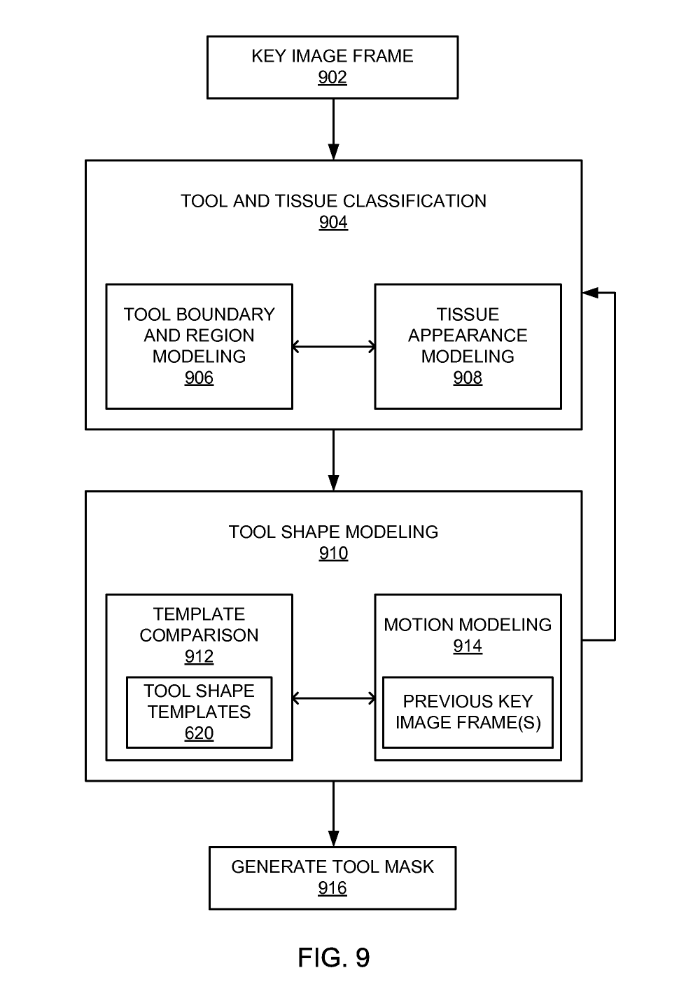

Referring now to FIG. 9, therein is shown an image processing flow chart detailing the tool isolation and tool shape modeling phases of operation. Beginning with step 902, a key image frame is obtained from the video taken by the camera 102 of FIG. 1. The key image frame can be a selected frame from a video stream—if the video is being taken at 60 fps, for example, the key image frame can be every fifth frame, but it is understood that the key image frame can be chosen based on the circumstances and equipment available.

At step 904, the key image frame is the input for the classification module of the processing unit 104 of FIG. 1. The key image frame is put through two complementary classification steps 906 and 908. In step 906, the key image frame undergoes segmentation through a segmentation module, inside the classification module. The segmented image frame undergoes boundary detection in a preliminary tool isolation process through the boundary detection module of the processing unit 104, coupled to the segmentation module and within the classification module. Areas within boundaries with characteristics such as straight lines, highly reflective surfaces (deviations from brightness of the rest of the key image frame), and uniform coloration can be used to calculate the potential tool outlines 518 of FIG. 5 using an outline generation module of the processing unit 104, coupled to the classification module.

At step 908, which can proceed in parallel with step 906, remaining regions of the key image frame are analyzed for consistency with human tissue. Known characteristics and databases of tissue models can be used by the tissue modeling module of the processing unit 104, inside the classification module and coupled to the boundary detection module, to confirm that regions of the key image frame which had not been marked as the potential tool outlines 518 are appropriately assigned as the background of interest 212 of FIG. 4, for example. Results from the boundary detection module and the tissue modeling module can be compared until the results largely match each other, ensuring greater accuracy. Once the results match, the potential tool outlines 518 can be finalized and further processed in step 910.

At step 910, the potential tool outlines 518 can be refined in a tool shape modeling process. The template comparison module of the processing unit 104, coupled to the outline generation module, can use provided examples in the tool shape templates 620 and compare the tool shape templates 620 with the potential tool outlines 518 in step 912. The template comparison module can estimate the pose (orientation of the surgical tools 210 of FIG. 4 relative to the camera 102) of the potential tool outlines 518 based on the boundaries detected and determine whether the potential tool outlines 518 match up with any of the tool shape templates 620, for example.

At step 914, a motion modeling process which can occur in parallel with step 912, the motion vector module of the processing unit 104, coupled to the template comparison module, can use the key image frame and a number of the previous key image frames to generate the motion vector overlay 722 of FIG. 7 by comparing the frames in chronological order and generating motion vectors from changes between frames. The motion tracking module of the processing unit 104, coupled to the motion vector module, can use motion vector data to generate the motion tracking layer 824 of FIG. 8.

The motion tracking layer 824, the motion vector overlay 722, and the potential tool outlines 518 can be combined and compared by the mask generation module of the processing unit 104, coupled to the motion tracking module, to generate the tool mask 314 of FIG. 3 in step 916. The mask generation module can facilitate cross-checking of the motion tracking layer 824 and the motion vector overlay 722 with the potential tool outlines 518 to ensure consistency of motion between different key image frames. The cross-checking can also help determine if the shape detected as one of the potential tool outlines 518 is moving as a rigid body (moving as a unit), and lead to more accurate generation of the tool mask 314, which can follow the surgical tools 210 as they move within the view of the camera 102. The tool mask 314 is used to block out the surgical tools 210 from calculations of exposure measure by an exposure module, coupled to the mask generation module, in order to obtain good quality for the image shown on the display interface 106 of FIG. 1.

The information used to generate the tool mask 314 can be fed back into step 904, refining and improving the tool and tissue classification process through a feedback module of the processing unit 104. For example, the motion modeling data generated by step 914 and the tool shape modeling data from in step 912 can be fed back into step 906 to speed up identification of likely locations for the surgical tools 210, and checked for consistency of motion from frame to frame (the surgical tools 210 should not jump around in the image frame, for example). Also for example, the motion tracking layer 824 and the motion vector overlay 722 can be used by a motion prediction module of the processing unit 104 to predict future motion of the surgical tools 210 to ensure that the tool mask 314 accurately follows the surgical tools 210 as they change positions from the current image frame to a future image frame.

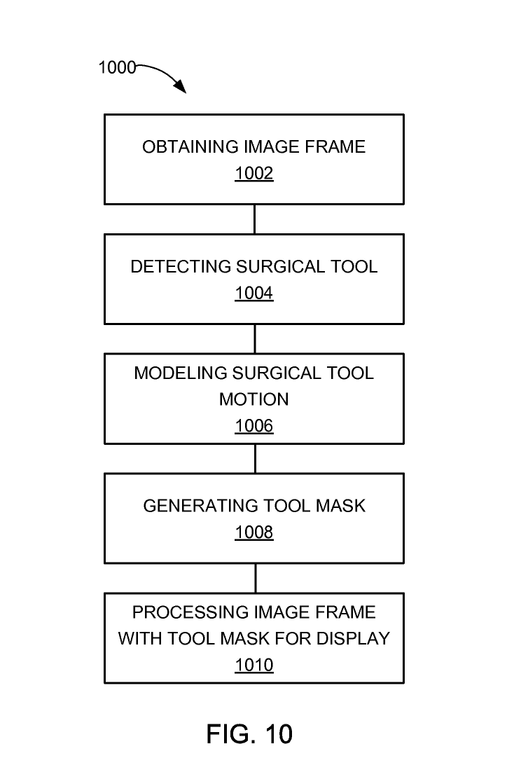

Referring now to FIG. 10, therein is shown a flow chart of a method 1000 of operation of a tool localization system in a further embodiment of the present invention. The method 1000 includes: obtaining an image frame with a camera in a block 1002; detecting a surgical tool in the image frame in a block 1004; modeling motion of the surgical tool in a block 1006; generating a tool mask, based on the surgical tool detected and the motion of the surgical tool, for covering the surgical tool in the image frame in a block 1008; and processing the image frame without the areas covered by the tool mask for display on a display interface in a block 1010.

The resulting method, process, apparatus, device, product, and/or system is straightforward, cost-effective, uncomplicated, highly versatile and effective, can be surprisingly and unobviously implemented by adapting known technologies, and are thus readily suited for efficiently and economically manufacturing tool localization systems/fully compatible with conventional manufacturing methods or processes and technologies.

Another important aspect of the present invention is that it valuably supports and services the historical trend of reducing costs, simplifying systems, and increasing performance.

These and other valuable aspects of the present invention consequently further the state of the technology to at least the next level.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

What is claimed is:

1. A method of operation of a tool localization system comprising:
obtaining an image frame with a camera;
detecting a surgical tool in the image frame;
modeling motion of the surgical tool based on the image frame and at least one prior image frame;
generating a tool mask, based on the surgical tool detected and the motion of the surgical tool, for covering the surgical tool in the image frame; and
processing the image frame without the areas covered by the tool mask for display on a display interface.

2. The method as claimed in claim 1 wherein obtaining the image frame with the camera includes obtaining the image frame with the camera and a light source.

3. The method as claimed in claim 1 wherein detecting the surgical tool includes:
segmenting the image frame;
detecting boundaries in the image frame;
generating a potential tool outline; and
correlating a tool shape template with the potential tool outline.

4. The method as claimed in claim 1 further comprising providing a processing unit connected to the camera.

5. The method as claimed in claim 1 wherein processing the image frame includes processing the image frame for exposure measure.

6. A method of operation of a tool localization system comprising:
providing a processing unit;
obtaining an image frame with a camera and a light source, the camera connected to the processing unit;
detecting a surgical tool in the image frame including:
segmenting the image frame,
detecting boundaries in the image frame,
generating a potential tool outline, and
correlating a tool shape template with the potential tool outline;
modeling motion of the surgical tool based on the image frame and at least one prior image frame;
generating a tool mask, based on the potential tool outline and the motion of the surgical tool, for covering the surgical tool in the image frame; and
processing the image frame for exposure measure without the areas covered by the tool mask for display on a display interface.

7. The method as claimed in claim 6 wherein modeling motion of the surgical tool includes:
generating a motion vector overlay; and
generating a motion tracking layer based on the motion vector overlay.

8. The method as claimed in claim 6 wherein detecting the surgical tool in the image frame includes detecting a background of interest in the image frame.

9. The method as claimed in claim 6 further comprising predicting future motion of the surgical tool for refining the position of the tool mask in a future image frame.

10. The method as claimed in claim 6 wherein modeling motion of the surgical tool includes determining a prioritized tracking sector.

11. A tool localization system comprising:
a camera for obtaining an image frame; and
a processing unit connected to the camera, the processing unit including:
a classification module for detecting a surgical tool in the image frame,
a motion vector module, coupled to the classification module, for modeling motion of the surgical tool based on the image frame and at least one prior image frame,
a mask generation module, coupled to the motion vector module, for generating a tool mask, based on the surgical tool detected and the motion of the surgical tool, for covering the surgical tool in the image frame, and
an exposure module, coupled to the mask generation module, for processing the image frame without the areas covered by the tool mask for display on a display interface.

12. The system as claimed in claim 11 further comprising a light source for providing light for the image frame.

13. The system as claimed in claim 11 wherein the processing unit includes:
a segmentation module of the classification module for segmenting the image frame;
a boundary detection module of the classification module, coupled to the segmentation module, for detecting boundaries in the image frame;
an outline generation module, coupled to the classification module, for generating a potential tool outline; and
a template comparison module, coupled to the outline generation module, for correlating a tool shape template with the potential tool outline.

14. The system as claimed in claim 11 wherein the processing unit is connected between and to the camera and the display interface.

15. The system as claimed in claim 11 wherein the exposure module is for processing the image frame for exposure measure.

16. The system as claimed in claim 11 further comprising:
a light source for providing light for the image frame;
wherein the processing unit is connected between and to the camera and the display interface, the processing unit including:
a segmentation module of the classification module for segmenting the image frame;
a boundary detection module of the classification module, coupled to the segmentation module, for detecting boundaries in the image frame;
an outline generation module, coupled to the classification module, for generating a potential tool outline;
a template comparison module, coupled to the outline generation module, for correlating a tool shape template with the potential tool outline; and
the exposure module is for processing the image frame for exposure measure.

17. The system as claimed in claim 16 wherein:
the motion vector module is for generating a motion vector overlay; and
further comprising:
a motion tracking module for generating a motion tracking layer based on the motion vector overlay.

18. The system as claimed in claim 16 wherein the classification module includes a tissue modeling module, coupled to the boundary detection module, for detecting a background of interest in the image frame.

19. The system as claimed in claim 16 further comprising a motion prediction module for predicting future motion of the surgical tool for refining the position of the tool mask in a future image frame.

20. The system as claimed in claim 16 wherein the motion tracking module is for determining a prioritized tracking sector.
